(19) United States
(12) Patent Application Publication
(10) Pub. No.: US 2024/0423475 A1
(43) Pub. Date: Dec. 26, 2024

(54) SKIN DIAGNOSIS SYSTEM

(71) Applicant: CHOWIS CO., LTD., Yongin-si Gyeonggi-do (KR)

(72) Inventor: Won Suk CHOI, Yongin-si Gyeonggi-do (KR)

(73) Assignee: CHOWIS CO., LTD., Yongin-si Gyeonggi-do (KR)

(21) Appl. No.: 18/701,349

(22) PCT Filed: Oct. 15, 2021
(86) PCT No.: PCT/KR2021/014409

§ 371 Date: May 16, 2024

(51) Int. Cl. A61B 5/00

(52) U.S. Cl. CPC A61B 5/0082; A61B 5/0013; A61B 5/0077; A61B 5/443; A61B 2576/02

(57) ABSTRACT

A skin diagnosis system includes a user terminal including a camera and a dedicated application, and a skin measurement device detachably provided on the user terminal. The skin measurement device includes a body part formed to be detachably provided on the user terminal, an optical part, which is arranged at the body part, includes one or more lenses, and adjusts, to the camera, measurement light reflected from a subject in a state in which the body part is mounted on the user terminal, a light source module, which includes a white light source portion for emitting white light and a UV light source portion for emitting UV light and radiates the white light or the UV light toward the subject, a communication part enabling communication with the user terminal, and a power source for providing power.

### TECHNICAL FIELD

Embodiments of the present disclosure relate to a skin diagnosis system.

### BACKGROUND ART

With the current increase in life expectancy, diagnosis and management of health conditions is one of essentials.

The skin, as an organ of the body encountering the external environment, contains a variety of metabolic products and components, and is sometimes an indicator of health conditions. In a specific example, a pale complexion may indicate poor circulation or a digestive disorder. In another example, when the skin breaks out, emotional stress may be suspected.

Conventionally, such skin conditions have usually been diagnosed by visual or tactile methods. Specifically, good or poor skin elasticity may be identified visually by optical microscopy, and the water content of the skin may be identified tactilely. However, such methods are not highly accurate and often rely on the experience of the person making the identification.

On the other hand, continuous skin monitoring for the diagnosis of health conditions is desirable. Accordingly, there is need for a skin condition diagnosis device and method capable of more easily performing monitoring and diagnosis without causing discomfort to a person being diagnosed. Accordingly, recent attempts have been made to diagnose skin conditions by radiating light onto the skin, capturing images of the skin, and analyzing the light information. However, most such diagnostic devices are expensive and thus are primarily used in skin care specialties such as skin care stores and dermatology clinics.

Therefore, there is need for a device which may accurately monitor the skin and may is easy to use.

### DISCLOSURE

### Technical Problem

Embodiments of the present invention seek to provide a skin diagnosis system that is portable, easy to use, and capable of accurately monitoring skin conditions.

### Technical Solution

As a means for realizing the technical solution described above, according to an embodiment of the present disclosure, provided is a skin diagnosis system including: a user terminal including a camera and a dedicated application; and a skin measuring device detachably provided on the user terminal, wherein the skin measuring device includes: a body part detachably provided on the user terminal; an optical part disposed on the body part and including one or more lenses to focus measurement light reflected from a subject on the camera in a state where the body part is mounted on the user terminal; a light source module including a white light source portion configured to emit white light and a UV light source portion configured to emit UV light, the light source module emitting the white light or the UV light toward the subject; a communication part capable of communicating with the user terminal; and a power supply configured to provide power, wherein the dedicated application is automatically activated when communication is connected in a state where the skin measuring device is mounted on the user terminal.

In an embodiment, the user terminal may generate an image capturing event signal when an image capturing event is detected in a state where the dedicated application is activated, and control the camera and the light source module using the dedicated application to obtain a plurality of diagnostic images for diagnosing the skin of the subject when the image capturing event signal is generated.

In an embodiment, the user terminal may extract analysis targets from the plurality of diagnostic images in a state where the dedicated application is activated, and calculate diagnostic scores for the analysis targets of the subject based on image information about the analysis targets and fundamental information about the subject.

In an embodiment, the diagnostic scores of the analysis targets may be calculated using one or more among an individual score of each of the analysis targets extracted from one or more images about the subject, the number of the analysis targets, distribution of the analysis targets, and fundamental information about the subject.

In an embodiment, the user terminal may obtain a facial image of the subject through the camera, identify an area corresponding to an eye in the obtained facial image of the subject, determine whether the eye is closed, and when the eye is determined to be closed, generate the image capturing event signal.

In an embodiment, the plurality of diagnostic images may include a first mode image, a second mode image, and a third mode image, and the user terminal sequentially obtains the first mode image, the second mode image, and the third mode image when the image capturing event signal is generated.

In an embodiment, the first mode image, the second mode image, and the third mode image may be images taken in different modes among UV mode, polarization mode, and white light mode.

In an embodiment, the body part may include a first body and a second body hinged to each other and facing each other, and may be detachably provided on the user terminal in a clip form.

In an embodiment, the skin measuring device may further include a sensor part configured to detect moisture of the subject.

In an embodiment, the user terminal may be configured to generate a notification using at least one of a speaker, a display, and a vibration device of the user terminal.

The above and other aspects, features, and advantages will be more clearly understood from the accompanying drawings, the claims, and the following detailed description.

### Advantageous Effects

According to the skin diagnosis system according to an embodiment of the present disclosure, it is portable, may be easily used, and may accurately monitor skin condition.

### DESCRIPTION OF DRAWINGS

FIG. 1 is a diagram schematically showing a skin diagnosis system according to an embodiment of the present disclosure.

FIGS. 2A and 2B are perspective diagrams of a skin measuring device according to an embodiment of the present disclosure.

FIG. 3 is a plan diagram of the skin measuring device of FIG. 2.

FIG. 4 is a front diagram of the skin measuring device of FIG. 2.

FIGS. 5A to 5C are diagrams showing a state where the skin measuring device according to an embodiment of the present disclosure is mounted on a user terminal. FIG. 6 is a block diagram schematically showing the user terminal of FIG. 1. FIG. 7 is a flowchart of the operation of the skin diagnosis system according to an embodiment of the present disclosure.

### MODE FOR INVENTION

Hereinafter, the following embodiments will now be described in detail with reference to the accompanying drawings, wherein identical or corresponding components are designated by the same reference numerals and repeated descriptions are omitted.

As the embodiments of the present disclosure are subject to various modifications, certain embodiments will be illustrated in the drawings and described in more detail in the detailed description. The effects and features of the embodiments of the present disclosure and methods for realizing the same will be clearly understood from the following description when taken in conjunction with the accompanying drawings.

However, the embodiments of the present disclosure are not limited to specific embodiments described hereinafter but may be embodied in a variety of different forms.

In the following embodiments, terms such as “first” and “second” are not used in a limiting sense, but only for the purpose of distinguishing one component from another component.

In the following embodiments, singular forms are intended to include plural forms, unless the context clearly indicates otherwise.

In the following embodiments, terms such as “comprise/include” or “have” indicate the presence of features or components described in the specification and do not exclude in advance the possibility of the addition of one or more other features or components.

In the following embodiments, it will be understood that when a portion such as a “unit”, “region”, or “component” is referred to as being positioned “on” or “above” another portion, the portion may be positioned on or above the other portion directly or indirectly via an intervening unit, region, component, or the like.

In the following embodiments, unless the context clearly indicates otherwise, terms such as “connect” or “couple” do not necessarily indicate a direct and/or fixed connection or coupling of two members and do not exclude the presence of other members interposed between the two members.

This indicates the presence of features or components described in the specification and does not exclude in advance the possibility of the addition of one or more other features or components.

In the drawings, the sizes of components may be exaggerated or reduced for ease of description. For example, because the size and thickness of each component shown in the drawings are arbitrary for ease of description, the following embodiments are not necessarily limited to the illustration.

Hereinafter, an embodiment of the present disclosure will be described in detail with reference to the accompanying drawings.

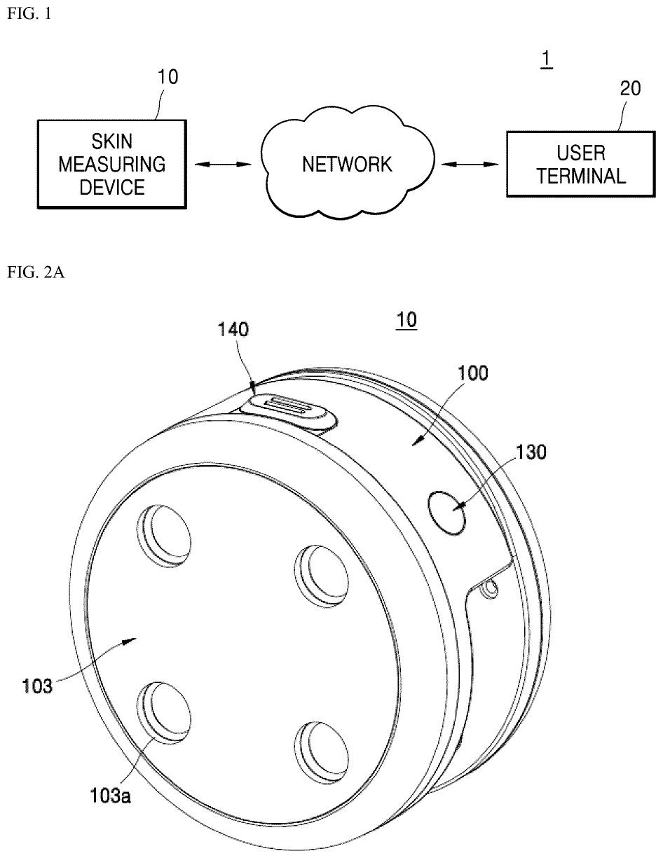

FIG. 1 is a diagram schematically showing a skin measurement system according to an embodiment of the present disclosure.

Referring to FIG. 1, the skin measurement system 1 according to an embodiment of the present disclosure may include a skin measuring device 10 and a user terminal 20. In addition, although not shown, the skin measurement system 1 may further include a plurality of user terminals 20 and a server (not shown) connected to the plurality of user terminals 20 through a network.

Here, the server (not shown) may be a server that collects image information of an analysis target obtained using each of the user terminals 20, fundamental information about a subject, and diagnosis information obtained by diagnosing the analysis target of the subject based on the image information and the fundamental information, processes the collected information, and provides the processed information to the user terminal 20. A plurality of servers (not shown) may be present depending on the amount of connection or data.

The skin measuring device 10 according to an embodiment of the present disclosure is provided in a form mountable on the user terminal 20 including a camera, and may measure skin conditions by operating in communication with the user terminal 20 while mounted on the user terminal 20. The skin measuring device 10 will be described later.

The user terminal 20 includes a camera, and a dedicated application for skin diagnosis may be provided. The user terminal refers to a communication terminal that may use web services in a wired or wireless communication environment. Here, the user terminal 20 may include a smartphone, a personal digital assistant (PDA), a portable multimedia player (PMP), navigation, a tablet PC, a kiosk, and the like.

The camera of the user terminal 20 may include a lens and an image sensor, and the image sensor may capture images of objects within an image capturing area using, for example, a complementary metal-oxide semiconductor (CMOS) module or a charge coupled device (CCD) module.

The input image frame is provided to the CMOS module or CCD module through the lens of the camera, and the COMS module or CCD module may convert an optical signal of an object that has passed through the lens into an electrical signal and output the same.

The user terminal 20 may include a storage medium capable of storing data converted from an image sensor. Here, the storage medium may include a magnetic storage medium or a flash storage medium, but the scope of the present disclosure is not limited thereto. These storage medium may include internal and/or external memory, volatile memory such as DRAM, SRAM, or SDRAM, non-volatile memory such as time programmable ROM (OTPROM), PROM, EPROM, EEPROM, mask ROM, flash ROM, and NAND flash memory, or NOR flash memory, a flash drive such as an SSD, a compact flash (CF) card, an SD card, a Micro-SD card, a Mini-SD card, an Xd card, or a memory stick, or a storage device such as an HDD.

The network is a connection structure that allows information exchange between the skin measuring device 10 and the user terminal 20, and includes a local area network (LAN), a wide area network (WAN), the Internet (WWW: World Wide Web), wired and wireless data communication networks, telephone networks, wired and wireless television communication networks, and the like.

Examples of the wireless data communication network include 3G, 4G, 5G, 3rd generation partnership project (3GPP), long term evolution (LTE), world interoperability for microwave access (WIMAX), Wi-Fi, Bluetooth communication, infrared communication, ultrasound communication, visible light communication (VLC), LiFi, and the like.

Hereinafter, a skin measuring device according to an embodiment of the present disclosure will be described in more detail with reference to the drawings.

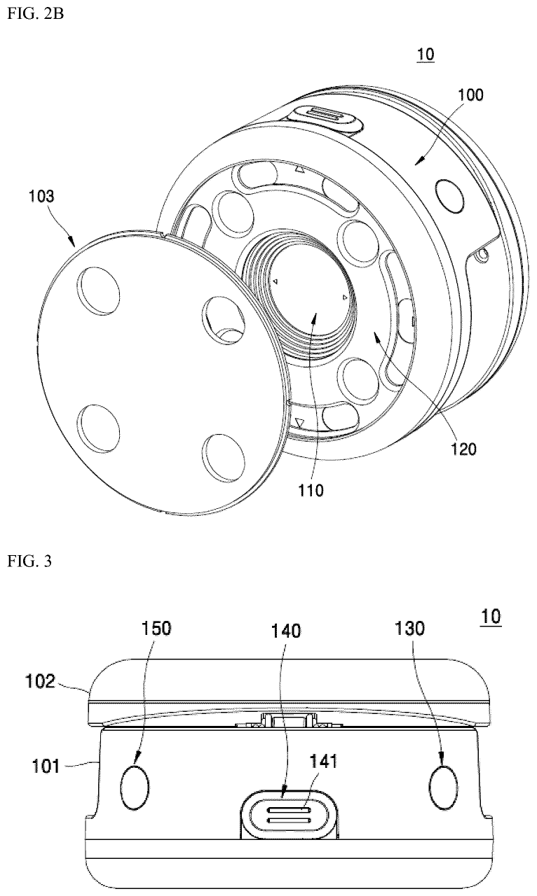

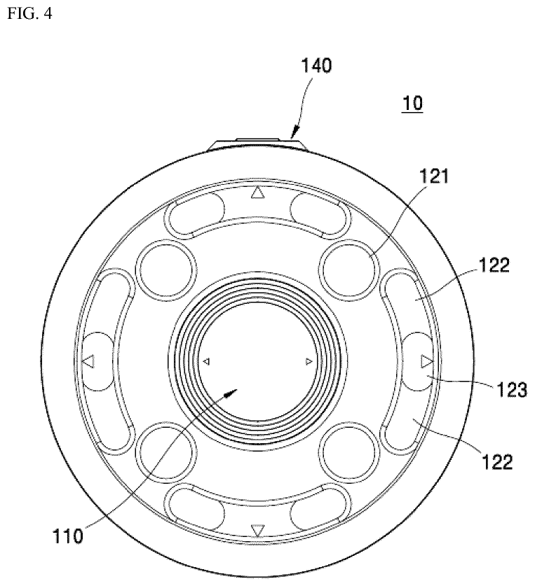

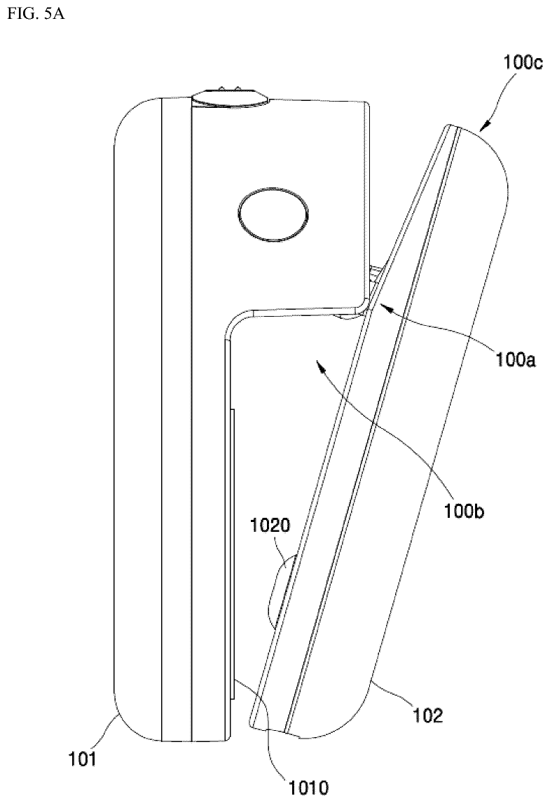

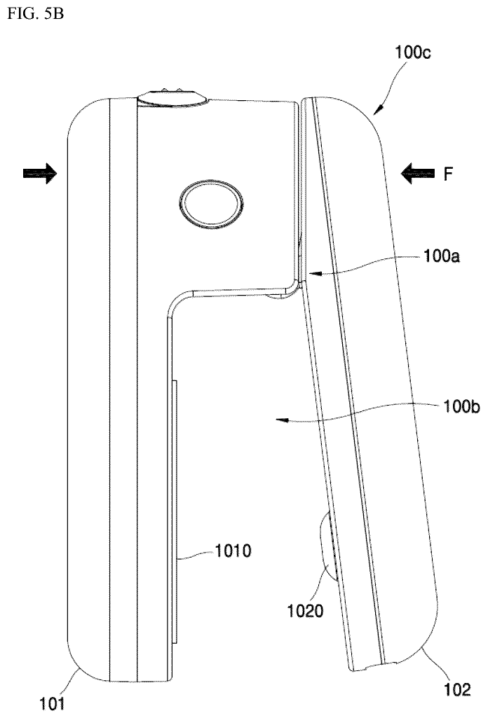

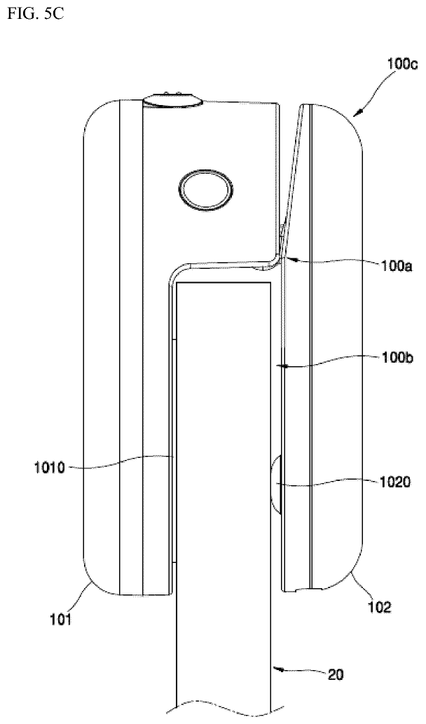

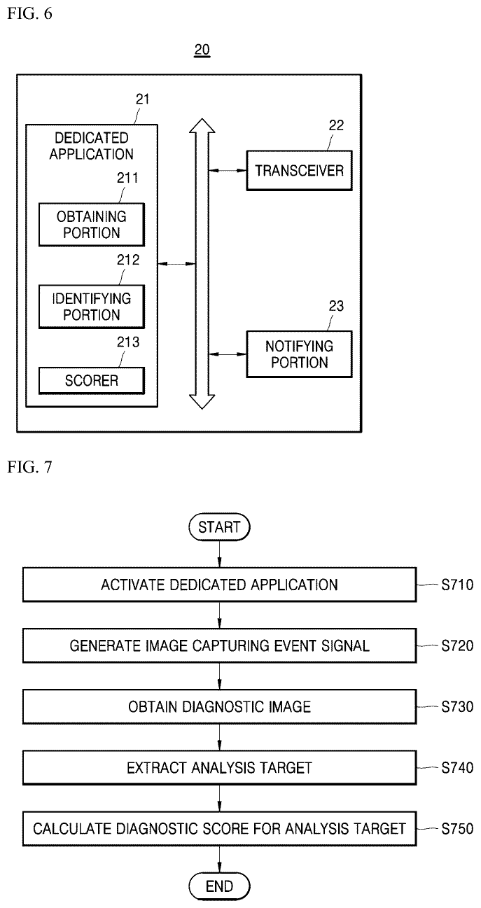

FIG. 2A is a perspective diagram of the skin measuring device 10 according to an embodiment of the present disclosure, and FIG. 2B is a diagram showing the cover 103 in a separated state from the skin measuring device 10 of FIG. 2A. FIG. 3 is a plan diagram of the skin measuring device 10 of FIG. 2, and FIG. 4 is a front diagram of the skin measuring device 10 of FIG. 2. In addition, FIGS. 5A to 5C are diagrams showing operations in which the skin measuring device 10 according to an embodiment of the present disclosure is mounted on the user terminal 20.

Referring to FIGS. 2 to 5, the skin measuring device 10 includes a body part 100, an optical part 110 provided on the body part 100, a light source module 120, and a switch part 130, a sensor part 140, and an illuminance sensor 150, as shown in the figures.

The body part 100 of the skin measuring device 10 according to an embodiment of the present disclosure forms an exterior as a housing of the skin measuring device 10, and is configured to enable the skin measuring device 10 to be mounted on a user terminal.

The body part 100 may be divided into two bodies 101 and 102, and a joint 100a to which the two bodies 101 and 102 are coupled may be provided.

To enable the skin measuring device 10 to be mounted on the user terminal, the body part 100 may be provided with gripping portions 100c which are portions configured to be held by the user and a mounting portion 100b into which the user terminal is inserted.

For example, the gripping portions 100c may be provided on the front and rear sides of the upper side of the body part 100, respectively, and may be provided on the two bodies 101 and 102, respectively.

The mounting portion 100b may be provided with buffers 1010 and 1020.

The buffers 1010 and 1020 are formed of a material such as silicone, and may improve adhesion between the skin measuring device and the user terminal inserted into the mounting portion 100b. In addition, scratches may be prevented from occurring on the surface of the user terminal during mounting.

For example, the body part 100 including the two bodies 101 and 102 may be provided with a mounting portion 100b between the two bodies 101 and 102, and the bodies 101 and 102 may be provided with buffers 1010 and 1020 formed of silicone, respectively.

The body part 100 may define an accommodation space to accommodate a PCB and a power supply therein.

In addition, the body part 100 may provide a space in which the optical part 110, the light source module 120, the switch part 130, and the sensor part 140 may be provided.

The skin measuring device 10 may include a circular cover 103 disposed on the front. The cover 103 may be disposed on the front of the body part 100 in a manner detachable from the body part 100 to protect the light source portions 121 to 123 and the optical part 110. In addition, the cover 103 may include a transparent material allowing light emitted from the light source portions 121 to 123 or light reflected from the subject and entering the optical part 110 to pass therethrough. In addition, a portion of the cover 103 corresponding to at least one of the optical part and the light source portions 121 to 123 may be provided with at least one through-hole 103a extending through the cover 103 so that light directed to the optical part 110 and the light source portions 121 to 123 may directly reach the subject without passing through the cover 103.

The optical part 110 of the skin measuring device 10 according to an embodiment of the present disclosure is disposed on the body part 100, and may include one or more lenses.

For example, the optical part 110 may include a wide-angle lens and a macro lens, and may include a fixing member capable of fixing the lenses.

The optical part 110 may magnify the surface image of the subject using the wide-angle lens and the macro lens, and may be fixed to the body part 100 to maintain a constant focal distance. The subject may be a part of the user's body, such as the face or an arm of the user, and may desirably be the skin of the user.

When the skin measuring device 10 is mounted on the user terminal, the optical part 110 may be positioned to correspond to the camera. Desirably, in a state where the body part 100 is mounted on the user terminal, the measurement light reflected from the subject may be focused on the camera of the user terminal. Accordingly, the measurement light reflected from the subject may be transmitted through the lens of the optical part 110 to the camera of the user terminal, thereby obtaining an image of the subject.

The light source module 120 of the skin measuring device according to an embodiment of the present disclosure may emit light, and may be disposed adjacent to the optical part 110.

The light source module 120 may include a plurality of light source portions spaced at regular intervals and arranged to surround the optical part 110, and in an embodiment, may include first light source portions 121, second light source portions 122, and third light source portions 123.

The first to third light source portions 121 to 123 may include light sources for emitting general light, UV light, and polarized light, respectively. For example, the first to third light source portions 121 to 123 may include a white light source for emitting white light, a UV light source for emitting UV light, and an LED light source in a specific wavelength band for emitting polarized light. White light may be light realized by a continuous spectrum across the entire visible spectrum, and UV light may be light corresponding to a wavelength in the range of about 10 to 400 nm. In addition, each of the third light source portions 123 for emitting polarized light may include a polarizing filter in addition to an LED light source.

In an embodiment, the first light source portions 121 may be arranged around the optical part 110 and spaced apart from each other at predetermined distances around the optical part 110. In addition, the second light source portions 122 and the third light source portions 123 may be configured and arranged as single units. For example, as shown in FIG. 4, each of the single units may include one second light source portion 122 and two third light source portions 123 disposed on both sides of the second light source portion 122.

The light source module 120 may control the operation of each of the light source portions 121 to 123 or the intensity of each of the light sources to diagnose skin conditions.

For example, coproporipyrin 3, i.e., one of the porphyrins produced in the skin by the bacterium responsible for acne vulgaris (Propionibacterium acne: P. acne), has an absorption spectrum with a peak at 407 nm. When light in the blue light band (400 to 440 nm) is applied to the skin, porphyrin strongly absorbs the energy of light in the blue wavelength band and fluoresces light in a unique wavelength band different from the absorbed wavelength. By analyzing this absorption spectrum, the skin condition of the subject may be diagnosed.

The sensor part 140 of the skin measuring device according to an embodiment of the present disclosure may include a sensor 141 to obtain more skin diagnosis information.

For example, the sensor part 140 may include a moisture sensor 141 detecting a moisture level of the subject. The moisture sensor 141 may be configured to measure changes in capacitance depending on moisture distribution in contact with the skin surface using a precision capacitor. In addition, the sensor part 140 may include an oil sensor (not shown) detecting an oil level of the subject and a PH sensor (not shown) measuring a PH level. The sensor part 140 may detect at least one a skin condition, a moisture level, an oil level, and a PH level of the subject and generate a measurement signal.

In order to prevent the sensor part 140 from being damaged when the skin measuring device is held to be mounted on the user terminal, the sensor part 140 may be provided on one side of the body part 100 independent of the part on which the skin measuring device is held. For example, the sensor part 140 may be provided on the top side of the body part 100.

The power supply of the skin measuring device according to an embodiment of the present disclosure may supply power to the sensor part 140 and the light source portions 121, and the switch part 130 may be configured to select an on/off state of the skin measuring device by connecting to or disconnecting from the power supply. The power supply may include a secondary battery such as a lithium-ion battery.

The illuminance sensor 150 of the skin measuring device 10 according to an embodiment of the present disclosure may be provided on one side of the body part 100, desirably, on the top side of the body part 100. Because the skin measuring device 10 may be used in a variety of environments, the illuminance sensor 150 may control the operation of each of the light source portions 121 to 123 or control the intensity of each of the light sources to respond to changes in illuminance due to the surrounding environment in order to prevent the consistency of the analysis from deteriorating depending on the environment. For example, when the illuminance of the environment in which the skin measuring device 10 is used is relatively high, the intensity of the light sources may be decreased through each of the light source portions 121 to 123, or when the illuminance of the environment in which the skin measuring device 10 is used is relatively low, the intensity of the light sources may be increased through each of the light source portions 121 to 123.

In addition, information about the illuminance measured by the illuminance sensor 150 may be transmitted to the user terminal through the network, and the illuminance of the environment in which the skin measuring device is used may be adjusted using the lighting device provided in the user terminal, based on the transmitted information about the illuminance.

FIGS. 5A to 5C are diagrams showing a state where the skin measuring device 10 according to an embodiment of the present disclosure is mounted on the user terminal 20, and referring to FIGS. 5A to 5C, the skin measuring device 10 according to an embodiment of the present disclosure may be detachably mounted on the user terminal 20.

For example, the skin measuring device 10 may be detachably provided on the user terminal 20 in the form of a clip.

At this time, the body part 100 may include a first body 101 and a second body 102. The first and second bodies 101 and 102 face each other, and may be hinged to each other at one side. The first and second bodies 101 and 102 may be relatively rotated around the hinged portions as an axis.

Referring to FIG. 5A, the first and second bodies 101 and 102 may be hinged to each other at the joint 100a. The joint 100a may be provided with an elastic member so that the skin measuring device may maintain the state as shown in FIG. 5A.

With respect to the joint 100a, the gripping portion 100c may be provided on one side, and the mounting portion 100b may be provided on the other side. The gripping portion 100c is a portion which is gripped by the user and to which force is applied. In addition, the mounting portion 100b is a portion into which the user terminal is inserted to be mounted thereto.

Referring to FIG. 5B, when a force greater than the elastic force of the elastic member is applied to the gripping portion 100c in a direction F, the gap between the first and second bodies 101 at the mounting portion 100b may be expanded due to the lever principle using the joint 100a as a fulcrum. A portion of the user terminal may be easily inserted into the mounting portion 100b provided between the first and second bodies 101 and 102 through the expanded gap.

Referring to FIG. 5C, when the force applied to the gripping portion 100c is removed, the elastic member provided in the connecting part 100a causes the gap between the first and second bodies 101 and 102 to return to the state as shown in FIG. 5A, and the user terminal may be mounted and fixed to the mounting portion 100b.

At this time, the first buffer 1010 of the first body 101 may support one side of the user terminal 20 while being in contact therewith, and the second buffer 1020 of the second body 102 may support the other side of the user terminal 20 while being in contact therewith.

The switch part may be provided separately on the body part 100, or may be provided on at least one of the first buffer 1010 and the second buffer 1020.

The switch part provided on the buffer 1010 and/or the buffer 1020 may be driven by changes in induced by the user terminal.

For example, the switch part provided on the buffer 1010 and/or the buffer 1020 may detect an increase in load applied to the buffer 1010 and/or the buffer 1020 by the user terminal in the state shown in FIG. 5C and operate so that the skin measuring device 10 is in an on state. In another example, in the state shown in FIG. 5A, when there is no load applied to the buffers 1010 and 1020, the operation may be performed so that the skin measuring device 10 is in an off state.

FIG. 6 is a block diagram schematically showing the user terminal 20 of FIG. 1.

Referring to FIG. 6, the user terminal 20 may include a dedicated application capable of working in conjunction with the skin measuring device 10.

The dedicated application may be configured to be automatically activated when the skin measuring device 10 is connected to the network while mounted on the user terminal.

The skin measuring device 10 may transmit and receive data to and from the user terminal 20 via the communication part.

As shown, the user terminal 20 may include a dedicated application 21, a transceiver 22, and a notifying portion 23.

The dedicated application 21 may obtain the image containing the subject received from the camera of the user terminal 20, and may perform an operation of extracting the analysis target from the image, an operation of calculating a diagnostic score for the analysis target of the subject based on image information about the analysis target and fundamental information about the subject, and the like.

Here, the analysis target is a diagnostic item indicating the skin condition of the subject, and may include, for example, pores, wrinkles, pigmentation, skin redness, sebum, dead skin cells, skin sensitivity, blackheads, and the like.

In addition, the fundamental information about the subject may include, for example, the age, gender, ethnicity information, past medical condition information, and/or diagnosis information of the subject, the medical condition information and/or diagnosis information of another subject group similar to the subject, the image of the subject, and the like. The dedicated application 21 may obtain the fundamental information about the subject through a questionnaire or, although not shown, through a database stored on a server.

The transceiver 22 may enable the user terminal 20 to connect to the skin measuring device 10 through the network, providing a communication interface necessary to provide transmission and reception signals between the user terminal 20 and the skin measuring device in the form of packet data.

The user terminal 20 may receive an on/off state signal of the skin measuring device 10 via the transceiver 22.

For example, when the skin measuring device is turned on with the power supply connected by the switch or turned off with the power supply disconnected by the switch, the user terminal 20 may transmit and receive a signal regarding the on/off state of the skin measuring device via the transceiver 22.

With respect to the signal received from the transceiver 22, the dedicated application 21 may be configured to be automatically activated in the user terminal 20 under predetermined conditions. For example, when the skin measuring device operates, the dedicated application 21 may be automatically activated by receiving a signal regarding the on state of the skin measuring device via the transceiver 22.

In addition, the user may directly activate the dedicated application 21 using a UI provided on the user terminal 20.

When an image capturing event is detected in a state where the dedicated application 21 is activated, the user terminal 20 may generate a signal for the image capturing event. The image capturing event may be detected, for example, by recognizing the closed state of an eye of the user, the voice of the user, pressing of a button provided on the user terminal, or the like.

The user terminal 20 may be configured to control the camera using the dedicated application 21 and automatically obtain a plurality of diagnostic images for diagnosing the skin of the subject when the image capturing event signal is generated.

At this time, the user terminal 20 may transmit a signal for controlling the light source module to the skin measuring device 10 via the communication part.

In response to the signal transmitted from the user terminal 20, the light source module 120 of the skin measuring device 10 may sequentially control the first light source portions 121, the second light source portions 122, and the third light source portions 123, thereby sequentially emitting white light, UV light, and polarized light. However, this is only an example, and the order of the emission of the light source module 120 may be changed.

According to an embodiment of the present disclosure, the dedicated application 21 may include an obtaining portion 211, an identifying portion 212, and a scorer 213.

The obtaining portion 211 of the dedicated application 21 may obtain one or more diagnostic images related to the subject through a camera based on the image capturing event signal generated by the user terminal 20.

Here, the diagnostic image related to the subject may be a full image of the subject, a partial image of the subject according to the image capturing position within the subject, or a partial image of the subject according to the order of image capturing time. For example, the diagnostic image may be a full image of the face of the user, a partial image taken from a portion of the face by the user using a photographing device, and the like.

In another example, the diagnostic image may include a first mode image, a second mode image, and a third mode image sequentially captured by the camera. At this time, each of the first mode image, the second mode image, and the third mode image may be an image taken in different modes among UV mode, polarization mode, and white light mode.

Polarization mode images may be captured using a polarizing film disposed on the optical part, and the polarizing film may be configured to be automatically disposed on the optical part using a motor.

The identifying portion 212 of the dedicated application 21 may identify an area corresponding to the eye of the user in the image of the subject detected by the camera. In addition, whether the eye of the user is closed or not may be determined from the identified image.

When the eye of the user is determined to be closed by the identifying portion 212, the user terminal 20 may generate an image capturing event signal and obtain a plurality of diagnostic images for diagnosing the skin of the subject by the obtaining portion 211 by using the dedicated application 21.

In an embodiment, when the image capturing event signal is generated, the user terminal 20 may transmit a control signal to control the light source module 120 of the skin measuring device 10 by using the dedicated application 21. The light source module 120 may control the first to third light source portions 121 to 123 so that white light, polarized light, and UV light are emitted sequentially at predetermined time intervals.

The scorer 213 of the dedicated application 21 may extract an analysis target from the obtained diagnostic image. Here, the analysis target may be a diagnostic item indicating the skin condition of the user, such as pores, wrinkles, pigmentation, and acne.

For example, when the analysis target is pores, the scorer 213 may generate image information of the pores including the pore area using a plurality of diagnostic images.

The image information of the analysis target may include size information of the analysis target, color information of the analysis target, depth information of the analysis target, and the like and, when the analysis target is, for example, pores, may mean information indicating the size of the pores, the color of the pores, the depth of the pores, and the like. The image information about the analysis target may include information about the number of analysis targets per unit area, distribution information of the analysis target throughout the subject, and the like

In addition, to generate status information of the subject contains moisture, the scorer 213 may contact the moisture sensor with the skin surface, measure a change in capacitance according to moisture distribution using a precision capacitor, and calculate the amount of moisture on the skin surface. In addition, state information of a new subject may be generated by comparing the state information of the generated subject with the fundamental information about the subject. At this time, the fundamental information about the subject may include information about the past state of the subject and/or information about the state of another subject group similar to the subject. Accordingly, the scorer 213 may generate a change in the state information of the subject as a result of comparing information about the current state of the subject and information about the past state of the subject, and may generate a change in the state information of the subject as a result of comparing the state information of the subject and the state information of another subject group similar to the subject. Here, both the state information of a new subject and a change in the state information of the subject may be included in the state information of the subject.

The scorer 213 may calculate a diagnostic score for the analysis target of the subject based on image information about the analysis target and the state information and the fundamental information about the subject. The scorer 213 may calculate a diagnostic score for the analysis target by applying fundamental information about the image information about the analysis target to predetermined analysis standards. Here, the analysis standards may include different equations or algorithms depending on the analysis target.

The diagnostic score of the analysis target may be calculated using one or more among individual scores of each of the analysis targets extracted from the diagnostic image, the number of the analysis targets, the distribution of the analysis targets, and fundamental information about the subject.

In an embodiment, when the analysis target is pores, the analysis criteria may calculate diagnostic scores using the score of first pores P1 having a size equal to or greater than a predetermined size, the score of second pores P2 having a size smaller than the predetermined size, and a diagnostic score using a first weight for the first pores P1, a second weight for the second pores P2, a third weight for the total number of the pores, and a fourth weight for the distribution of the pores. Here, in addition to the first to fourth weights, the analysis standards may further include a fifth weight for the fundamental information about the subject.

The scorer 213 may classify the first pores P1 having a size equal to or greater than a predetermined size and the second pores P2 having a size smaller than the predetermined size from the image information about the analysis target, and calculate the score for the first pores P1 using depth information of the classified first pores P1, the average size of the first pores P1, color information of the first pores P1, and the like. Similarly, the scorer 213 may calculate the score for the second pores P2 using depth information of the classified second pores P2, the average size of the second pores P2, the color information of the second pores P2, and the like. The above description is only an example of pores as an analysis target, and when the analysis target is wrinkles, or when the analysis target is pigmentation or acne, the diagnostic score may be calculated based on other analytic criteria.

The diagnostic score may be represented as a number from 1 to 99 or 5 levels of evaluation.

On the other hand, the notifying portion 23 of the user terminal 20 may be configured to generate a notification using at least one of a speaker, a display, and a vibration device of the user terminal 20.

For example, when it is determined by the identifying portion 212 that the eyes of the user are not closed, the user terminal 20 may generate at least one of audible, tactile, and visual signals using a device provided on the user terminal 20.

FIG. 7 is a flowchart of the operation of the skin diagnosis system according to an embodiment of the present disclosure.

As shown, in operation S710, a dedicated application may be activated. The dedicated application may be activated by receiving a signal about the on state of the skin measuring device via the transceiver when the skin measuring device is in operation, or the user may directly activate the dedicated application using the UI provided on the user terminal.

In operation S720, when the dedicated application is activated, an image capturing event signal may be generated. The image capturing event signal may be generated by the user terminal, for example, by recognizing the closed eye of the user, by detecting the voice of the user, or in response to pressing the button provided on the user terminal.

In operation S730, when the image capturing event signal is generated, a diagnostic image may be obtained. The diagnostic image may be at least one of a UV mode image, a polarization mode image, and a white light mode image for diagnosing the skin of the subject.

In operation S740, when a diagnostic image is obtained, an analysis target may be extracted from the diagnostic image. Here, the analysis target may be a diagnostic item indicating the skin condition of the user, such as pores, wrinkles, pigmentation, and acne.

In operation S750, when the analysis target is extracted, a diagnosis score for the analysis target may be calculated. The diagnostic score of the analysis target may be calculated using one or more among individual scores of each of the analysis targets extracted from the diagnostic image, the number of the analysis targets, the distribution of the analysis targets, and fundamental information about the subject. The calculated diagnostic score may be represented on the display of the user terminal as a number from 1 to 99 or 5 levels of evaluation.

In the above description, operations S710 to S750 may be further divided into additional operations or combined into fewer operations according to an embodiment of the present disclosure. In addition, some operations may be omitted as required, and the order between operations may be changed.

The present disclosure has been described hereinabove with reference to the exemplary embodiments illustrated in the drawings, but it will be apparent to a person having ordinary skill in the art that the embodiments are illustrative only, and that various modifications and variations of the embodiments are possible. Accordingly, the true technical scope of protection of the present disclosure shall be defined only by the technical idea of the appended claims.

1. A skin diagnosis system comprising:
a user terminal comprising a camera and a dedicated application; and
a skin measuring device detachably provided on the user terminal,
wherein the skin measuring device comprises:
a body part detachably provided on the user terminal;
an optical part disposed on the body part and comprising one or more lenses to focus measurement light reflected from a subject on the camera in a state where the body part is mounted on the user terminal;
a light source module comprising a white light source portion configured to emit white light and a ultraviolet (UV) light source portion configured to emit UV light, the light source module emitting the white light or the UV light toward the subject;
a communication part capable of communicating with the user terminal; and
a power supply configured to provide power, and
wherein the dedicated application is automatically activated when communication is connected in a state where the skin measuring device is mounted on the user terminal.

2. The skin diagnosis system of claim 1,
wherein the user terminal generates an image capturing event signal when an image capturing event is detected in a state where the dedicated application is activated, and controls the camera and the light source module by using the dedicated application to obtain a plurality of diagnostic images for diagnosing the skin of the subject when the image capturing event signal is generated.

3. The skin diagnosis system of claim 2,
wherein the user terminal extracts analysis targets from the plurality of diagnostic images in a state where the dedicated application is activated, and calculates diagnostic scores for the analysis targets of the subject based on image information about the analysis targets and fundamental information about the subject.

4. The skin diagnosis system of claim 3,
wherein the diagnostic scores of the analysis targets are calculated using one or more among an individual score of each of the analysis targets extracted from one or more images about the subject, the number of the analysis targets, distribution of the analysis targets, and fundamental information about the subject.

5. The skin diagnosis system of claim 2,
wherein the user terminal obtains a facial image of the subject through the camera, identifies an area corresponding to an eye in the obtained facial image of the subject to determine whether the eye is closed, and generates the image capturing event signal when the eye is determined to be closed.

6. The skin diagnosis system of claim 2,
wherein the plurality of diagnostic images comprise a first mode image, a second mode image, and a third mode image, and
the user terminal sequentially obtains the first mode image, the second mode image, and the third mode image when the image capturing event signal is generated.

7. The skin diagnosis system of claim 6,
wherein the first mode image, the second mode image, and the third mode image are images taken in different modes among an UV mode, a polarization mode, and a white light mode.

8. The skin diagnosis system of claim 1,
wherein the body part comprises a first body and a second body which are hinged to each other and face each other, thereby being detachably provided on the user terminal in a clip form.

9. The skin diagnosis system of claim 1,
wherein the skin measuring device further comprises a sensor part configured to detect moisture of the subject.

10. The skin diagnosis system of claim 1,
wherein the user terminal is configured to generate a notification using at least one of a speaker, a display, and a vibration device of the user terminal.
